(19) United States
(12) Patent Application Publication
(10) Pub. No.: US 2005/0053971 A1
(43) Pub. Date: Mar. 10, 2005

(54) Methods and constructs for developing cell lines for biological assays

(76) Inventors: Alex Chenchik, Emerald Hills, CA (US); Alex Munishkin, Santa Cruz, CA (US)

Correspondence Address: MOSER, PATTERSON & SHERIDAN, L.L.P., 3040 POST OAK BOULEVARD, SUITE 1500, HOUSTON, TX, 77056, US

(21) Appl. No.: 10/836,179

(22) Filed: Apr. 30, 2004

Related U.S. Application Data
(63) Continuation-in-part of application No. 10/658,632, filed on Sep. 8, 2003.
(63) Continuation-in-part of application No. 10/766,605, filed on Jan. 27, 2004.

(51) Int. Cl. C12Q 1/68; G01N 33/53

(52) U.S. Cl. 435/6; 435/7.1

(57) ABSTRACT

The present invention provides methods and compounds for high throughput analysis of transcription regulation using transcription reporter libraries. The present invention is a high throughput system that allows rapid development of cell lines that can be used in assays to screen for virtually any biological function or phenotype of interest.

### RELATED APPLICATIONS

This is a continuation-in-part of copending application Ser. No. 10/658,632, filed on Sep. 8, 2003 and copending application Ser. No. 10/766,605 filed on Jan. 27, 2004.

### FIELD OF THE INVENTION

The present invention is a high throughput system that allows rapid development of cell lines that can be used in assays to screen for virtually any biological function or phenotype of interest.

### BACKGROUND OF THE INVENTION

The study of the interactions between DNA and proteins is one of the most rapidly growing areas of molecular biology. Transcription factors, a subset of DNA binding proteins, play a central role in the regulation and control of gene expression, replication, and recombination. Because of these important roles, inhibition and stimulation of transcription factor binding to DNA is of great interest in the discovery of potential new drug targets.

An enormous body of work generated over the past three decades has revealed that eukaryotic gene transcription is a remarkably intricate biochemical process that is tightly regulated at many levels. Biochemical and genetic analysis of various model organisms has identified an astounding number of protein factors responsible for transcriptional control. The expression and activity of transcription factors may be regulated by numerous intra- and extra-cellular signals. Moreover, transcription factor activity may be regulated by interaction with other proteins, by catalyzing modifications, participating in processing or transport, etc. Therefore, various combinations of transcription factors associated with a particular gene can elicit many different patterns of gene expression. Although a large assortment of gene-specific DNA-binding regulators was anticipated in the art, the sheer complexity of the general machinery, relative to prokaryotes, has been a surprise. Even more unexpected are the numerous, intricate layers of control imposed by the diversification of co-activators and co-repressors, some of which possess enzymatic activity. Many interactions between the transcription factors that have been identified to date have been discerned. Despite these advances, however, little is known about the detailed mechanisms by which individual genes are turned on or off in a cell.

Several different protocols have been developed to study DNA-protein interactions or protein-protein interactions, such as DNA-protein photocrosslinking, the electrophoretic mobility shift assay, in vivo reporting systems like the one- and two-hybrid yeast expression systems, and transcription and reporter vectors. However, for the most part these assays allow for investigation of only one transcription factor per reaction. In addition, the methods currently known in the art are useful for identifying high-abundance transcription factors; however, they are not useful for identifying low-abundance factors that may play a role equally as important as the high-abundance factors. Essentially, the methods for analyzing transcription factors currently in use are time consuming and can be very expensive, especially when a large number of factors are to be analyzed.

There is a desire in the art for improved compositions and methods for analyzing transcription factors, as well as for constructing reporter cell lines to be used in assays for biological processes. The present invention satisfies these needs in the art.

### SUMMARY OF THE INVENTION

The present invention is directed to innovative, rapid and robust transcriptional reporter methods for performing analysis of transcription regulation. Specifically, the present invention is directed to embodiments of a library of transcription factor response sequences that allow for analysis of several to tens of thousands of transcription factors in one assay; thus providing the first, true high-throughput method for analyzing the functional activity of transcription factors. The transcription factor response sequences may be natural sequences, consensus sequences (sequences constructed from several similar sequences) or sequences predicted from natural sequences. Essentially, the present invention is a high throughput system that allows rapid development of cell lines that can be used in assays to screen for virtually any biological function or phenotype of interest.

Thus, one embodiment of the present invention provides a transcription reporter library comprising at least ten different transcriptional reporter vector constructs. Each construct comprises an identification sequence coupled to at least one transcription factor response sequence operably coupled to a first promoter to produce transcription factor response cassettes. Each transcription factor response cassette is different from one another and each then is operably coupled to first reporter sequence. In preferred aspects of this embodiment, the identification sequences are different from one another. Also in a preferred aspect of this embodiment of the invention, the identification sequence is downstream of the first promoter.

In yet another embodiment of the present invention there is provided a transcription reporter library comprising at least ten different transcriptional reporter vector constructs, where each construct comprises at least one transcription factor response sequence operably coupled to a first promoter to produce transcription factor response cassettes. Each transcription factor response cassette is different from one another and is operably coupled to an identification sequence and a first reporter sequence.

In some aspects of either of these embodiments of the invention, the transcription reporter vector constructs further comprise a second reporter gene operably coupled to a second promoter. In some aspects, the first and/or second reporter sequences code for a bioluminescent, chemiluminescent or fluorescent protein. If a second reporter sequence is present, the second promoter may be a constitutive promoter. Also, in some aspects of this embodiment of the present invention, the first reporter sequence is a part of a viral vector, and preferably the viral vector is a lentiviral vector.

In other aspects of either of these embodiments of the present invention, the transcription factor response sequences comprise one to ten transcription response element sequences, attenuator sequences, enhancer sequences, silencer sequences or other response sequences; and the transcription factor response sequences comprise two or more copies of the same transcription response element sequence, attenuator sequence, enhancer sequence, silencer sequence or other response sequence, or a combination of one or more of any of transcription response element sequences, attenuator sequences, enhancer sequences, silencer sequences or other response sequences. Also, in preferred aspects, the transcription reporter construct is packaged in viral particles.

One embodiment of the present invention provides a method for constructing reporter cell lines for screening for a biological process of interest comprising transducing target cells with a viral transcription reporter library of the present invention; treating the target cells with an effector; analyzing the target cells for altered reporter activity where altered reporter activity indicates a change in a biological process; and selecting one or more target cells with the altered reporter activity.

In yet another embodiment of the present invention, there is provided a method for identification of sequences that affect transcription of a reporter gene in response to an effector, comprising transducing target cells viral particles containing the transcription reporter constructs of the present invention; treating the target cells with an effector; analyzing the target cells for altered reporter activity; selecting one or more target cells with the altered reporter activity; isolating the identification sequence nucleic acid from the selected target cells; and identifying the transcription factor response sequence present in the target cells. The transcription factor response sequence controls the transcription of the reporter gene and is responsive to a transcription factor, which is in turn responsive to the effector.

In yet another embodiment, the present invention provides a method for analyzing differences in activities of effectors in at least two different cell populations comprising transducing at least two different cell populations with viral particles containing the transcription reporter constructs of the present invention; analyzing the at least two cell populations for reporter activity; selecting cell fractions with altered levels of reporter activity within each population; isolating the identification sequence nucleic acid from the selected cells from each cell population; identifying the transcription factor response sequence present in the selected cells from each cell population based on the identification sequence or by direct sequencing of the transcription response sequence. The transcription factor response sequence controls the transcription of the reporter gene and is responsive a transcription factor, which is in turn responsive to the effector; and comparing the identified transcription factor response sequences affected in each cell population.

Other embodiments of the present invention provide methods for identification of sequences that affect transcription of a reporter gene in response to an effector, comprising transducing target cells with viral particles containing the transcription reporter constructs of the present invention; treating the target cells with an effector; analyzing the target cells for altered reporter activity where altered reporter activity indicates a change in transcription; selecting one or more target cells with the altered reporter activity; isolating the identification sequence nucleic acid from RNA from the selected target cell; and identifying the transcription factor response sequence present in the target cell, where the transcription factor response sequence controls the transcription of the reporter gene and is responsive to a transcription factor, which is in turn responsive to the effector.

Yet another embodiment of the present invention provides methods for analyzing differences in activities of effectors in at least two different cell populations comprising transducing target cells with viral particles containing the transcription reporter constructs of the present invention; analyzing each cell population for reporter activity; selecting cell fractions with altered levels of reporter activity within each population; isolating the identification sequence nucleic acid from RNA from the selected cells from each cell population; identifying the transcription factor response sequence linked to the identification sequence present in the selected cells from each cell population, where the transcription factor response sequence controls the transcription of the reporter gene which is responsive to a transcription factor, which is in turn responsive to the effector, and comparing the identified transcription factor response sequences affected in each cell population.

Another embodiment of the present invention provides a method for simultaneously assaying in the same reaction at least five transcription factors that affect transcription of a reporter, comprising obtaining at least five identification sequences; coupling each of the at least five identification sequences to at least five different transcription factor response sequences to produce a set of transcription factor response cassettes; ligating the set of transcription factor response cassettes into a reporter vector to produce transcription reporter constructs; packaging the transcription reporter constructs into viral particles to produce a viral reporter library; transducing target cells with the viral reporter library; treating the target cells with an effector; analyzing the target cells for altered reporter activity where altered reporter activity indicates a change in transcription; selecting one or more target cells with the altered reporter activity; isolating the identification sequence nucleic acid from the selected target cells; and identifying the transcription factor response sequence present in the target cell, where the transcription factor response sequence controls the transcription of the reporter gene and is responsive to a transcription factor, which is in turn responsive to the effector. In some aspects of this embodiment, a first analyzing step performed between the transducing step and the treating step, and an amplification step is performed between the isolating step and the identifying step. In preferred embodiments, the identifying step is performed by hybridization of the isolated identification sequence coupled to the transcription factor response element to a microarray.

In addition, the present invention provides a method of constructing a packaged viral transcription reporter library comprising synthesizing a set of at least 100 different identification sequences on a glass substrate; decoupling the at least 100 different identification sequences from the glass substrate; synthesizing transcription factor response sequences on a glass substrate; decoupling the transcription factor response sequences from the glass substrate; coupling each identification sequence to at least one transcription factor response sequence to produce at least 100 different transcription factor response cassettes; obtaining a reporter vector, wherein the reporter vector comprises viral sequences and at least one reporter sequence; ligating the transcription factor response cassettes to the reporter vector to produce viral response constructs, such that the reporter vector's first reporter sequence is operably coupled to a first promoter and the transcription factor response cassette; and packaging the viral transcription reporter construct in viral particles; thereby producing a packaged viral transcription reporter library. In preferred aspects of this embodiment, the reporter further vector comprises a second reporter gene operably coupled to a second promoter, and the transcription factor response sequences comprise one to ten transcription response element sequences, attenuator sequences, enhancer sequences, silencer sequence or other response sequences, where the one to ten copies sequences are two or more copies of the same transcription response element sequence, attenuator sequence, enhancer sequence, silencer sequence or other response sequence, or a combination of one or more of any of transcription response element sequences, promoter sequences, enhancer sequences, silencer sequence or other response sequences coupled to an identification sequence.

In yet another embodiment of the present invention, there is provided a method for screening for transcription factors capable of altering the transcription level of a reporter gene comprising introducing into cells a transcription reporter library comprising at least ten different identification sequences each of which is coupled to at least one transcription factor response sequence operably coupled to a promoter and a reporter sequence; screening the cells for a cell or subset of cells exhibiting an altered level of reporter gene transcription, wherein the altered phenotype is due to the action of a transcription factor on the transcription factor response sequence. In some aspects of this embodiment, an additional step of is performed, comprising identifying the transcription factor response sequence on which the transcription factor responsible for the altered phenotype acted.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIGS. 1A and 1B show simplified flow charts showing two methods for constructing transcription reporter constructs according to one embodiment of the present invention.

FIGS. 2A-D are simplified block constructs of various embodiments of the transcription factor response cassettes coupled to a reporter gene according to the present invention. FIG. 2E is an example of a transcription factor response cassette comprising an identification sequence (underlined) and a transcription factor response sequence for p53 (overlined). The sequences in lower case are common sequences that are used to amplify the identification sequence/transcription factor response sequence cassette.

FIG. 3 illustrates a more detailed design of a transcription reporter construct according to one embodiment of the present invention.

FIG. 4 is a schematic of one method to produce a recombinant viral library according to embodiments of the present.

FIG. 5 shows constructs useful in one embodiment for producing a transcription reporter viral library according to certain embodiments of the present invention.

FIG. 6 is a simplified flow chart of one embodiment of a method for using the transcription reporter libraries according to the present invention.

FIG. 7 is a schematic of the method of FIG. 6.

FIG. 8 is a simplified flow chart of another method for using the transcription reporter libraries according to the present invention.

### DETAILED DESCRIPTION

In the following description, numerous specific details are set forth to provide a more thorough understanding of the present invention. However, it will be apparent to one of skill in the art that the present invention may be practiced without one or more of these specific details. In other instances, well-known features have not been described in order to avoid obscuring the present invention.

The present invention is directed to innovative, rapid and robust methods and libraries for performing analysis of transcription regulation and transcription factor response sequences. The transcription factor response sequences may be natural sequences, consensus sequences (sequences constructed from several similar sequences) or sequences predicted from natural sequences.

Generally, conventional methods of molecular biology, microbiology, recombinant DNA techniques, cell biology and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

A “vector” is a replicon, such as plasmid, phage, cosmid or viral construct to which another DNA segment may be attached.

A “transcription factor response sequence” is a nucleic acid sequence that responds to or binds to a transcription factor or another protein regulating transcription, including but not limited to, transcription response elements (TREs), enhancers, attenuators or silencers. A transcription factor response sequence may comprise a single TRE, attenuator, enhancer, silencer or other response sequence; two or more copies of the same TRE, attenuator, enhancer, silencer or other response sequence; or a combination of one or more of any of TREs, attenuators, enhancers, silencers or other response sequences.

A “promoter sequence” is a DNA regulatory region capable of binding RNA polymerase in a cell and initiating transcription of a downstream sequence.

The term “reporter gene” refers generally to a gene that codes for the expression of a polynucleotide or polypeptide capable of acting as a label or that is easy to detect and/or quantity. The term “reporter vector” refers to a genetic construct comprising one or more reporter genes.

The term “identification sequence” or “ID sequence” refers to oligomers comprising natural, modified or analog nucleotides. Typically, in embodiments of compositions and methods according to the present invention, ID sequences are chosen so that they are different from one another, and do not hybridize to sequences complementary to one another.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA may or may not be stably integrated into the genome of the cell. With respect to eukaryotic cells, a stably transformed cell is one in which the transforming DNA has become integrated into a host cell chromosome or is maintained extra-chromosomally so that the transforming DNA is inherited by daughter cells during cell replication. Such a stably transformed eukaryotic cell is able to establish cell lines or clones comprised of a population of daughter cells containing the transforming DNA.

The term “effector” refers to virtually anything that might impact a cell, tissue, organ or organism, including but not limited to, environmental conditions such as temperature, growth media (salts, nutrients, etc.), aging, amount or wavelength of light, atmospheric conditions, time of day and the like; chemical compounds, such as vitamins, salts, carcinogens, toxic agents, drugs, polymers, proteins, enzymes, antibodies and the like; genetic agents, such as constructs expressing proteins, siRNAs, aptamers, ribozymes, antisense RNAs or proteins, siRNAs, aptamers, ribozymes, antisense RNAs delivered directly to the cell, organ or organism; and any other chemical, genetic or physical condition.

The term “microarray” refers to arrays or ordered arrangements of different targets, such as proteins, peptides or nucleic acids on a solid or semi-solid or porous support such as a slide, membrane, polymer layer attached to solid support, chip, bead, or microwell plate where the location of each target is known. Targets can be bound to a support by photolithographic techniques, phosphoramidite chemistry, photochemistry, electrochemistry, covalent or non-covalent immobilization or other methods known in the art.

The present invention, in one embodiment, provides a transcription reporter library. The transcription reporter constructs used for the library according to the present invention can be generated synthetically or enzymatically by a number of different protocols known to those with skill in the art, and the constructs may be purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research. In a preferred embodiment, the transcription factor response cassette or separate cassette components thereof are synthesized using phosphoramidite chemistry. For synthesis of large numbers of oligos, one or more components of the transcription factor response cassettes are synthesized on a microarray glass surface or other support surface using photolithography, pin or ink-jet deposition, electrochemical or acoustic delivery or other protocols well known in the art. Synthesis of oligos on an array surface is preferable if the number of oligos is more than 1,000, particularly if the number of oligos synthesized exceeds 10,000 or even 20,000. Synthesis via array on a glass substrate is, among other features, cost effective and allows for flexibility of cassette design changes.

The reporter vector comprising the reporter gene sequence(s) may utilize a viral or non-viral backbone. The choice of vector will depend on the type of cell in which propagation is desired and the purpose of propagation. Certain vectors are useful for amplifying and making large amounts of the desired transcription reporter constructs. Other vectors are suitable for expression of the reporter gene in cells in culture or directly in model organisms. The choice of appropriate vector is well within the skill of the art, and many such vectors are available commercially. Viral vectors, preferably retroviral vectors, and more preferably lentiviral vectors, may be used in the transcription factor response reporter libraries of the present invention. To prepare the transcription factor response reporter constructs, the transcription factor response cassette is inserted into the reporter vector typically by means of DNA ligase attachment to a cleaved restriction enzyme site in the reporter vector or by protocols known in the art, including use of recombination enzymes such as the Cre-lox system, in-fusion PCR, etc.

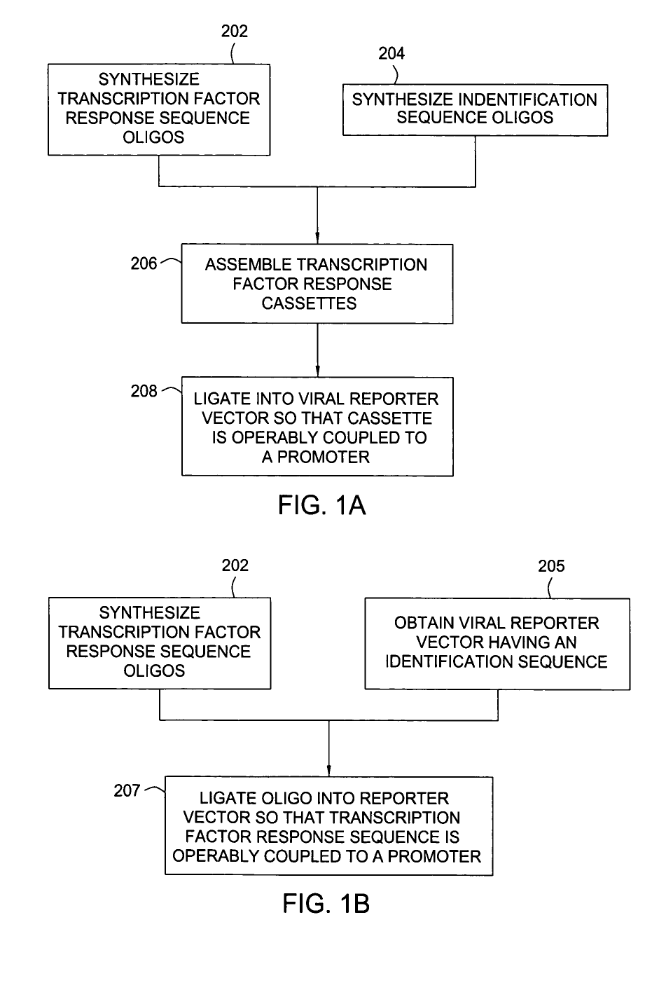

FIG. 1A shows a simplified flow chart showing the steps of one embodiment of one method for constructing the transcription factor reporter constructs of the present invention. In step 202, transcription factor response sequence oligomers are synthesized, preferably on a glass substrate. At step 204, ID sequence oligomers are synthesized, also preferably on a glass substrate. The transcription factor response cassettes are assembled by coupling the transcription factor response sequences with the appropriate ID sequences at step 206, and this cassette is then ligated into a viral reporter construct. In some embodiments of the present invention, the transcription factor response cassette also contains a promoter to drive expression of the reporter gene in the reporter vector. The promoter sequence may be synthesized as part of the transcription factor response sequence component of the cassette, the ID sequence component of the cassette, or may be synthesized as a separate component. Alternatively, the entire transcription factor response sequence/promoter sequence/ID sequence component may be synthesized as one long oligomer. In yet another embodiment, the promoter may be part of the reporter vector.

The transcription factor response sequence oligos and the ID sequence oligos can be synthesized and ligated by methods known in the art. For example, each different transcription factor response sequence oligo and each different ID sequence can be synthesized separately. Then each specific transcription factor response sequence is combined with a specific ID sequence and ligated together to form the transcription factor response cassette (i.e., a plurality of ligation reactions). In another embodiment, transcription factor response oligos may be synthesized with a “tag” having a specific sequence, while the ID sequence oligos are synthesized with “tags” having complementary tag sequences. In this embodiment the various transcription factor response oligos and the various ID sequence oligos may be combined and ligated in one reaction, as the ID sequence oligo tags will hybridize only to a complementary tag from a specific transcription factor response oligo. Combinations of these embodiments may also by employed

FIG. 1B shows an embodiment where the transcription factor response sequences are synthesized at 202, and viral reporter vectors having an identification sequence as part of the reporter vector are obtained at 205. At step 207, the oligo with the transcription factor response sequence is ligated to the reporter vector. As with the embodiment in FIG. 1A, the promoter sequence may be a component of the transcription factor response sequence oligo or a component of the reporter vector. In this embodiment, the ID sequence may be a component of the transcription factor response sequence oligo (to form a transcription factor response cassette), or the ID sequence may be a component of the reporter vector.

The transcription reporter construct comprises at least one transcription factor response sequence. The transcription factor response sequence may be any transcription factor response nucleotide sequence, including, but not limited to, transcription response elements (TREs), attenuators, enhancers or silencers. A transcription factor response sequence may comprise a single TRE, attenuator, enhancer, silencer or other response sequence; two or more copies of the same TRE, attenuator, enhancer or silencer or other response sequence; or a combination of one or more of any of TREs, attenuators, enhancers, silencers or other response sequences.

Any protein that participates in the initiation or affects the efficiency of transcription is a transcription factor. The present invention identifies transcription factors that act directly by recognizing cis-acting sites on DNA such as promoters, response elements, attenuators, silencers or enhancers or other response sequences or parts thereof; and by recognizing trans-acting factors such as factors that act indirectly on the factors which then act on promoters, response elements, attenuators, silencers, enhancers or other response sequences. For example, transcription response elements are specific, short consensus elements located upstream (up to 200 bp or more) of the transcription start point. Enhancers are the regions of DNA that may be a kilobase or more away from a gene they control, and may be located upstream, downstream or even within a gene they control. Silencers are control regions of DNA that, like enhancers, may be located a kilobase or more away from a gene they control. However, when transcription factors bind to silencers, expression of the gene they control is repressed. There are other, less-characterized response sequences in genomic DNA which control transcription of specific genes, like nuclear matrix-attachment sites which work at distances even greater than a kilobase.

Although there are common types of peptide motifs and protein types found in transcription factors, such as helix-turn-helix motifs, zinc-finger motifs, leucine zipper motifs and helix-loop-helix motifs, AP1-like proteins, Myogenic bHLH proteins and Myc family proteins, there are over 500 different transcription factors that have been identified to date, and possibly many hundreds more that have not. Some factors may bind only one, specific transcription factor response sequence; but other factors are known to bind many transcription factor response sequences where the transcription factor response sequences may or may not have common motifs, and vary in length and sequence aside from the motif. For example, p53 is known to bind 70-75 different transcription factor response sequences. It is conceivable that a factor may bind tens or even hundreds of transcription factor response sequences. Moreover, p53 is a member of a family that also includes the p63 and p73 transcriptional factors that bind to some of the same transcription factor response sequences as p53 but have different affinities, levels of regulation, levels of expression, etc. Estimating that there may be 1000 transcription factors, and that an average transcription factor can bind 20 transcription factor response sequences, the total number of transcription factor response sequences could be upward of 20,000. To add complexity, many different transcription factors may bind at the same time to form a complex, the transcription factor response sequences may overlap with one another, and/or one transcription factor response sequence may bind more than one transcription factor. The libraries of the present invention allow investigation of 20,000 or more such transcription factor response sequences in a single assay.

The transcription factor response sequences according to embodiments of the present invention may comprise one to ten TRE sequences, attenuator sequences, enhancer sequences, silencer sequences or other response sequences; two or more copies of the same TRE sequence, attenuator sequence, enhancer sequence, silencer sequence or other response sequence; or a combination of one or more of any of TRE sequences, attenuator sequences, enhancer sequences, silencer sequences or other response sequences. In a preferred embodiment, the total number of separate TRE sequences, attenuator sequences, enhancer sequences, silencer sequence or other response sequences in the transcription factor response sequence is three to nine. Also, in a preferred embodiment, the transcription factor response sequence contains three to nine copies of the same TRE sequence, attenuator sequence, enhancer sequence, silencer sequence or other response sequence. In a more preferred embodiment, the total number of separate TRE sequences, attenuator sequences, enhancer sequences, silencer sequence or other response sequences in the transcription factor response sequence is four to six. Also, in a more preferred embodiment, the transcription factor response sequence contains four to six copies of the same TRE sequence, attenuator sequence, enhancer sequence, silencer sequence or other response sequence or any combination thereof.

As described in the description of FIGS. 1A and 1B, the transcription factor response sequence optionally is part of a transcription factor response cassette that comprises an identification (ID) sequence. The ID sequences of the present invention are oligomers comprising natural, modified or analog nucleotides that allow for identification of the transcription factor response sequence in each cassette. Theoretically, the transcription factor response sequences themselves could be used for identification (by sequencing or hybridization for example), but use of ID sequences allows for high throughput design optimization. For example, ID sequences may be relatively short oligomer sequences because such oligomer sequences are easily amplified by PCR or sequenced. Alternatively, or in addition, ID sequences may be amplified and then used as hybridization probes, optimizing detection of the ID sequences and corresponding transcription factor response sequence by microarray.

To design ID sequences, rules well known in the art are applied, such as excluding sequences with strong secondary structure or self-complementarity (for example long hairpins), very high (more than 70%) or very low (less than 40%) GC content, long stretches of more than six consecutive identical bases, long stretches of sequences enriched in certain motifs, long stretches of purines or pyrimidines, stretches of particular base repeats, and the like. In addition, applying the rules that have been developed in the art for selection of PCR primers and hybridization probes allows for selection of ID sequences that do not cross-hybridize with one other. Commercial computer programs exist that allow for choosing oligomer sequences based on criteria chosen by the user.

For example, Primer3 is a computer program that selects PCR primers for a variety of applications, selects single primers for sequencing reactions, and designs oligonucleotide hybridization probes. In selecting oligos for primers or hybridization probes, Primer3 considers many factors, including oligo melting temperature, length, GC content, 3′ stability, estimated secondary structure, the likelihood of annealing to or amplifying undesirable sequences (for example interspersed repeats), the likelihood of primer-dimer formation between two copies of the same primer, and the accuracy of the source sequence. Another commercial program, PrimerSelect, is a suite of tools for the design and analysis of oligonucleotides, including primers for PCR, sequencing, probe hybridization and transcription. Using DNA, RNA or back-translated proteins as templates, PrimerSelect details thermodynamic properties for annealing reactions. The software lists all possible primers, ranked in order of suitability. Oligo is a multi-functional program that searches for and selects oligonucleotides from a sequence file for PCR, sequencing, site-directed mutagenesis, and various hybridization applications. Oligo calculates hybridization temperature and secondary structure of oligonucleotides based on the nearest neighbor change in free energy values.

In a preferred embodiment of ID sequence selection, the homology cut-off is set for less than 80%, and, in a more preferred embodiment, the homology cut-off is set for less than 75% or even 70% using the standard default criteria for the oligo BLAST algorithm. In addition, ID sequences with similar melting temperatures or thermodynamic stabilities are selected, to provide similar performance in microarray analysis. In addition, the ID sequences that are selected preferably do not have homology to any sequence entry in GenBank related to eukaryotic organisms. Even more preferably, the ID sequences that are selected do not have homology to any sequence entry in GenBank known to relate to any phage, virus, archaebacteria, or prokaryotic or eukaryotic organism.

As described previously, the transcription factor response cassette may comprise a promoter, preferably an RNA polymerase II or III promoter, for expression of the reporter gene. Alternatively, the reporter vector into which the transcription factor response cassette is inserted may contain the promoter. Whether in the cassette or as part of the reporter vector, the RNA Polymerase promoter is positioned so as to be operably linked to the reporter gene once the transcription factor response cassette is inserted into the reporter vector.

The promoter linking the transcription factor response sequence to the reporter gene preferably is a constitutive promoter, such as the promoter for ubiquitin, CMV, β-actin, histone H4, EF-1-alfa or pgk controlled by RNA polymerase II, or the U6 snRNA, H1 snRNA or tRNA promoters controlled by RNA polymerase III. Even more preferably, the promoter linking the transcription factor response sequence to the reporter gene preferably is a minimal constitutive promoter such as mCMV, which does not work efficiently without active transcription factor response sequences, but is activated if one or more transcription factors bind a transcription factor response sequence. In another embodiment, a constitutive promoter works at a high basal level but stops transcription if a transcription factor binds to silencer-type TRE.

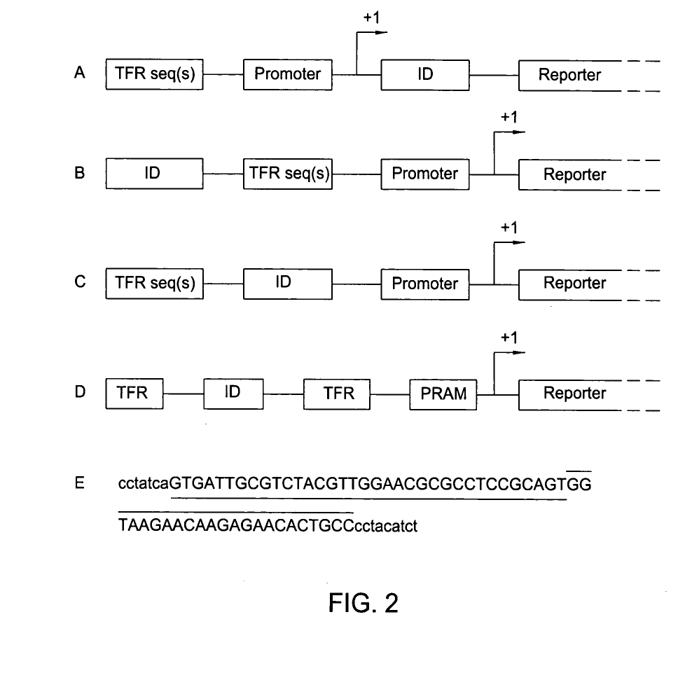

FIGS. 2A-C demonstrate three alternatives for placement of the promoter, ID sequence, transcription factor response sequence and the reporter gene. In FIG. 2A, the ID sequence is placed downstream of the promoter. This is a preferred embodiment, as it allows for transcription of the ID sequence into RNA, thereby allowing for the identification of the ID sequence by isolating RNA from the selected cells. Moreover, the presence of reporter gene may not then be necessary, as the expression level of the ID sequence could define promoter activity. As stated before, expression level of an ID sequence could be profiled by hybridization with a microarray comprising sequences complementary to the ID sequences.

In FIG. 2B, the ID sequence is located upstream of the transcription factor response sequence and the promoter. In FIG. 2C, the ID sequence is located between the transcription factor response sequence and the promoter. If the embodiments shown in FIGS. 2B and 2C are used in conjunction with the lentiviral vector described herein, the ID sequence is identified by isolating, and preferably amplifying, genomic DNA from the selected cells. FIG. 2D is an example of an ID sequence (underlined) and a transcription factor response sequence (overlined). The sequences in lower case are common sequences that are used for amplifying the cassette. In addition, because in this embodiment the common sequences are used to amplify both the ID sequence and the transcription factor response sequence, the amplification product can be used for repeat cloning and another round of transduction, and selection as described infra.

Once constructed, the transcription factor response cassette is then ligated into a reporter vector. In a preferred embodiment, the reporter vector comprises at least one reporter gene. This reporter gene becomes operably linked to the transcription factor response sequence once the transcription factor response cassette is ligated into the reporter vector. The reporter gene(s) used in the reporter vector of the present invention preferably are any polynucleotide or polypeptide moiety that may be expressed in the target cell or organism. Preferred reporter genes are those that code for gene products that can be quickly and easily detected and quantified such as bioluminescent, chemiluminescent or fluorescent proteins. Alternatively, reporter genes may be nucleic acid or amino acid sequences that can be identified by hybridization to an array (such as the ID sequence when placed downstream of the promoter). Disclosure for such reporters may be found in U.S. Pat. No. 5,422,266 to Cormier et al.; U.S. Pat. No. 5,541,309 to Tsien et al; U.S. Pat. No. 5,418,155; to McElroy et al.; U.S. Pat. No. 5,583,024 to Harpold et al., and U.S. Pat. No. 5,491,084 to Chalfie et al.; the disclosure for the pCop-green C vector at www.evrogen.com; and see Zhang, et al., Nature 3:906 (2002). In one embodiment of the present invention, a green fluorescent protein from a copepod, pCop-greenC, is the reporter gene driven by the transcription factor response cassette.

In addition to the first reporter gene, the reporter vector optionally comprises a second reporter gene under the control of a second promoter. The second reporter gene serves as a control for determining the extent of the alteration of transcription of the first reporter gene. The second promoter is preferably a constitutive promoter, such as those described above. The second reporter gene can be any reporter useful for detecting presence of the reporter vector. Optionally, the first and second reporter products can be detected by the same or complimentary methods such as by fluorescence-activated cell sorting (FACS). In one embodiment, the second reporter gene is the gene for a red fluorescent protein from jellyfish, jelly RFP (again see www.evrogen.com).

In one preferred embodiment of the present invention, the reporter construct used for high efficiency transduction or transfer and expression of the reporter genes in various target cell types is derived from a retrovirus. Retroviruses are any virus belonging to the family Retroviridae, comprising single-stranded RNA animal viruses characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In a more preferred embodiment of the present invention, the reporter construct used for high efficiency transduction or transfer and expression of the reporter genes in various target cell types is derived from a lentivirus. Lentiviruses are members of the retrovirus family. Lentivirus vectors are often pseudotyped with Vesicular Stomatitus Virus G Protein (VSV-G). Lentiviruses include the human immunodeficiency virus (HIV), the etiologic agent of the human acquired immunodeficiency syndrome (AIDS); visan-maedi, which causes encephalitis (visna) or pneumonia in sheep; the caprine arthritis-encephalitis virus, which causes immune deficiency, arthritis, and encephalopathy in goats; equine infectious anemia virus (EIAV), which causes autoimmune hemolytic anemia and encephalopathy in horses; feline immunodeficiency virus (FIV), which causes immune deficiency in cats; bovine immune deficiency virus (BIV) which causes lymphadenopathy and lymphocytosis in cattle; and simian immunodeficiency virus (SIV), which causes immune deficiency and encephalopathy in non-human primates. Vectors that are based on HIV retain <5% of the parental genome, and <25% of the genome is incorporated into packaging constructs, which minimizes the possibility of the generation of revertant replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Since lentivirus infects virtually 100% of dividing and resting cells, transcription factor response reporter libraries are introduced into target cells efficiently and selection is not required. Similar high efficiency transfer of transcription reporter libraries to dividing cells may be achieved with retroviral vectors. Most importantly, the uniformity of expression from retroviral inserts and the lack of significant position effect allow one to work with polyclonal populations of cells yet avoid the problem of clonal variability. In addition, the approach can be applied to cells within a living organism, as the reporter constructs easily can be introduced by injection or other delivery means into organs, blood, tumors, or embryos.

Reverse transcription of the retroviral RNA genome occurs in the cytoplasm. Unlike C-type retroviruses, the lentiviral cDNA, complexed with other viral factors (known as the pre-initiation complex), is able to translocate across the nuclear membrane and transduce non-dividing cells. A structural feature of the viral cDNA—a DNA flap—seems to contribute to efficient nuclear import. This flap is dependent on the integrity of a central polypurine tract (cPPT) that is located in the viral polymerase gene, so, in preferred embodiments, the lentiviral-derived constructs of the present invention retain this sequence. Lentiviruses have broad tropism, low inflammatory potential, and result in an integrated vector construct. The main limitations are that integration might induce oncogenesis in some applications. The main advantage to the use of lentiviral vectors is that gene transfer is persistent in most tissues or cell types.

A viral transcription reporter construct according to one embodiment of the present invention comprises sequences necessary for the production of recombinant retrovirus in a packaging cell, expression of the reporter genes, and contains the ID and transcription factor response sequences. Generation of the viral construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. The viral construct may incorporate sequences from any known organism that makes the construct useful in the methods of the present invention. The sequences may be incorporated in their native form or they may be modified. For example, the sequences may comprise insertions, deletions or substitutions. In a preferred embodiment, the viral construct comprises sequences from an FIV genome.

The viral reporter construct preferably comprises sequences from the 5′ and 3′ LTRs of a lentivirus. More preferably, the viral construct comprises the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Preferably the LTR sequences are FIV LTR sequences. The virus also can incorporate sequences for MMLV or MSCV, RSV or mammalian genes.

The viral reporter construct preferably comprises an inactivated or self-inactivating 3′ LTR. The 3′ LTR may be made self-inactivating by any method known in the art. In the preferred embodiment the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome comprises an inactivated 5′ LTR, preventing later excision of the provirus sequence from the target cell.

Optionally, the U3 sequence from the lentiviral 5′ LTR may be replaced with a promoter sequence in the viral construct. This may increase the titer of virus recovered from the packaging cell line. An enhancer sequence may also be included. Also, the viral construct preferably is cloned into a plasmid that may be transfected into a packaging cell line. The preferred plasmid preferably comprises sequences useful for replication of the plasmid in bacteria.

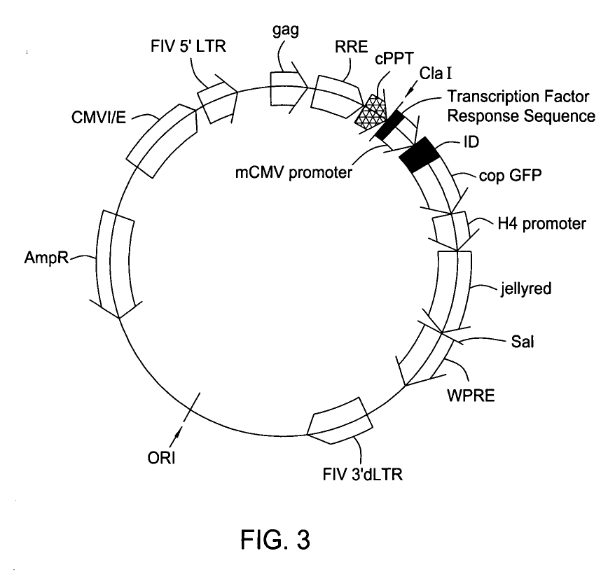

FIG. 3 shows one embodiment of a transcription reporter construct vector according to the present invention and contains genetic elements for transduction, packaging, stable integration of the transcription reporter construct into genomic DNA, and expression of the reporter sequences; though many of the sequences shown are optional such as the cPPT, RPE, WPRE, etc. From the 12-o'clock position, there is an FIV gag gene, followed by a Rev response element (which participates in transduction and integration of the viral construct), followed by a sequence coding for the central polypurine tract (cPPT). After the cPPT, a ClaI site marks the beginning of the transcription factor response cassette. In the embodiment shown in FIG. 3, the most 5′ component of the cassette is the transcription factor response sequence, followed by a minimal CMV promoter (mCMV), followed by the ID sequence. Following the ID sequence is the pCop-greenC reporter sequence that is driven by the transcription factor response cassette, followed by an H4 promoter driving the jelly RFP sequence. Next, there is a WPRE element (which provides the viral with transcript greater stability), an FIV 3′ LTR that is disabled, an origin of replication, an ampicillin resistance gene, a CMV promoter fused with a 5′ LTR (instead of a U3 region), and, finally an FIV LTR. Other sequences that may be included in the transcription reporter construct are those coding for auxiliary proteins such as those responsible for viral transduction, packaging, like orf, Rev, CTE, poly (A) signals, poly (A) enhancers, IRES and the like.

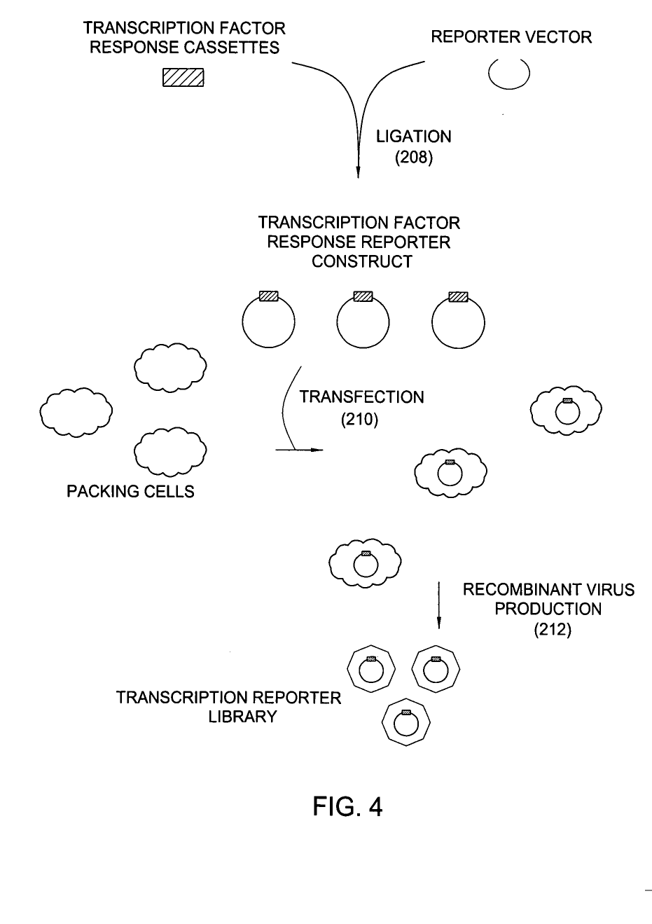

Any method known in the art may be used to produce infectious retroviral particles whose genome comprises an RNA copy of the viral construct described herein. In one embodiment, the viral construct is introduced into a packaging cell line, where the packaging cell line expresses in trans the viral proteins that are required for the packaging of the viral genomic RNA into viral particles. The packaging cell line may be any cell line that is capable of expressing retroviral proteins, including 293, HeLa, D17, MDCK, BHK, CᵣFK and Cf2Th. Such a packaging cell line is described, for example, in U.S. Pat. No. 6,218,181, and Heiser, W., Gene Delivery to Mammalian Cells, Volume 2, Viral Gene Transfer Techniques (2000); Federico, M., Lentivirus Gene Engineering Protocols (2004); and Machida, C., Viral Vectors for Gene Therapy Methods and Protocols (2003), and more such packaging cell lines are developed as the art progresses. FIG. 4 is a schematic of one method of producing recombinant virus according to the present invention. First, transcription reporter cassettes are ligated into a reporter vector to produce a transcription reporter construct (step 208). The transcription reporter construct is then transfected into packaging cells (step 210), and the transfected cells then produce recombinant virus containing the transcription reporter construct; i.e., the transcription reporter library (step 212).

Alternatively, a cell line that does not code for or express viral proteins may be transiently transfected with one or more constructs comprising nucleic acids that encode the necessary viral proteins. In such an embodiment, a cell line that does not stably express the necessary viral proteins is co-transfected with two or more plasmids. One of the plasmids comprises the viral transcription factor response reporter construct. The other plasmid(s) comprises nucleic acids encoding the proteins necessary to allow the cells to produce functional virus that is able to infect the desired host cell.

The packaging cell line may not express vector-specific envelope gene products. In this case, the packaging cell line will package the viral genome into particles where a viral-vector-specific envelope protein is replaced with other envelope proteins. As the envelope protein is responsible, in part, for the host range of the viral particles, the viruses of the library of the present invention preferably are pseudotyped. A “pseudotyped” retrovirus is a retroviral particle having an envelope protein that is from a virus other than the virus from which the RNA genome is derived. The envelope protein may be from a different retrovirus or a non-retrovirus. One envelope protein of particular use in the present invention is the vesicular stomatitius virus G (VSV-G) protein. Thus, the packaging cell line preferably is transfected with a plasmid comprising sequences encoding a membrane-associated protein, such as VSV-G, that will permit entry of the virus into a target cell. One with skill in the art can choose an appropriate pseudotype for the target cell used. In addition to conferring a specific host range, a chosen pseudotype may permit the virus to be concentrated to a very high titer. Viruses alternatively can be pseudotyped with ecotropic envelope proteins that limit infection to a specific species or can be pseudotyped to recognize other proteins, like antibodies or ligands that bind to specific receptors or proteins on the surface of target cells.

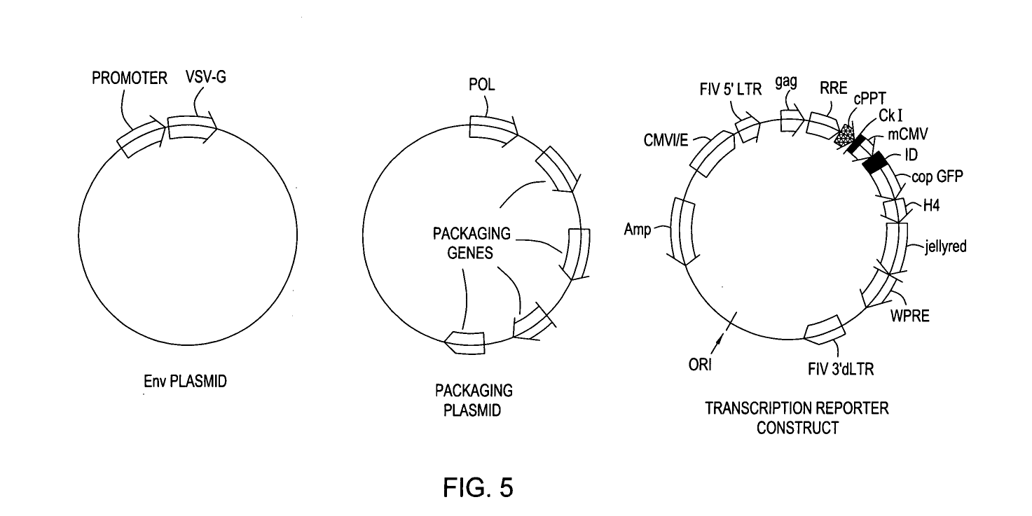

FIG. 5 shows one embodiment of a multi-vector system for performing the methods of the present invention. A first vector is a plasmid comprising the VSV-G (env) sequence. A second vector, the packaging plasmid, comprises genes necessary for transcription and packaging of an RNA copy of the transcription reporter construct into recombinant viral particles. The third vector is the transcription reporter construct shown in FIG. 3, and contains genetic elements for transduction, packaging, stable integration of the transcription reporter construct into genomic DNA, and expression of the reporter sequence(s). It should be understood by those with skill in the art that this is merely one embodiment of a vector system. Many other embodiments may be employed. For example, the env gene may be included on the packaging vector or the transcription factor response reporter construct in a two-vector system. Alternatively, additional regulatory genes or genes that facilitate packaging, transduction and/or expression of the viral construct may be carried on the transcription factor response reporter vector.

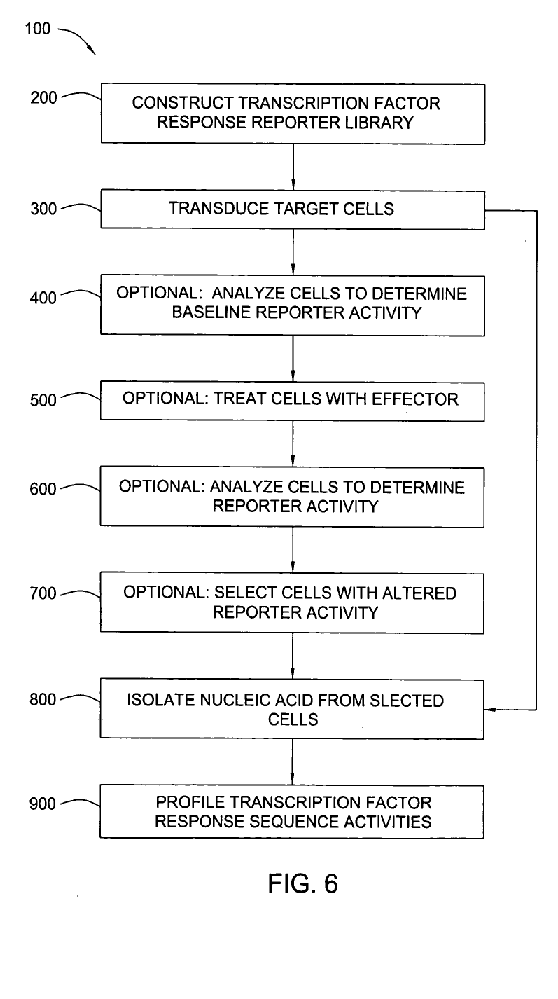

FIG. 6 is a block diagram of a simplified method 100 according to one embodiment of the present invention. In a first step, a transcription reporter library is constructed (step 200). Next, target cells of interest are transduced with the transcription reporter library (step 300). In an optional step, the transduced cells are then analyzed to determine a baseline reporter activity at step 400. Also optionally, the target cells may then treated with an effector (step 500), and the treated cells may then again analyzed to detect reporter activity (step 600). Target cells having altered reporter activity optionally are then selected (step 700). At step 800, nucleic acid is isolated from the selected cells (optionally) or the entire cell population. Finally at step 900, the transcription factor response sequence responsible for the altered reporter activity is identified.

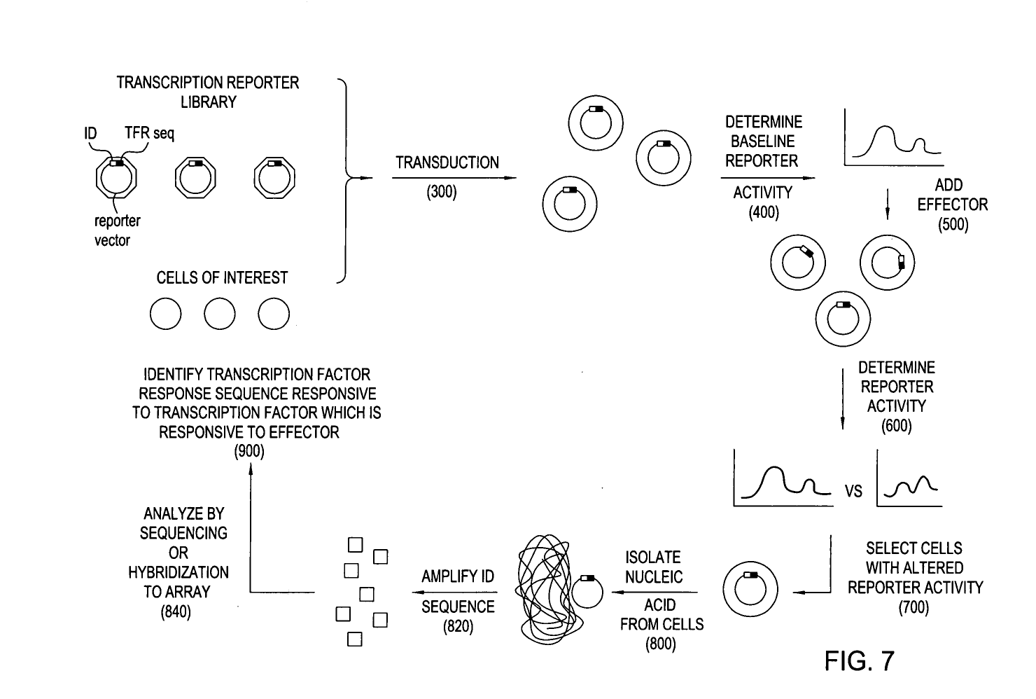

FIG. 7 is a schematic of one embodiment of the method shown in FIG. 6. First, cells of interest are transduced with the transcription factor response library (step 300). The baseline reporter activity is determined at step 400, and then an effector is added at step 500. The level of reporter activity is determined again (600), and treated cells with altered reporter activity are selected (700). Nucleic acids are isolated from the selected cells (800), and the ID sequences are, preferably, amplified (step 820). Finally, the ID sequences are analyzed by sequencing, or, preferably, by hybridization to a microarray (step 840), allowing for identification of the ID sequence, and, thus, the transcription factor response sequence responsive to a transcription factor, which in turn is responsive to the effector (900).

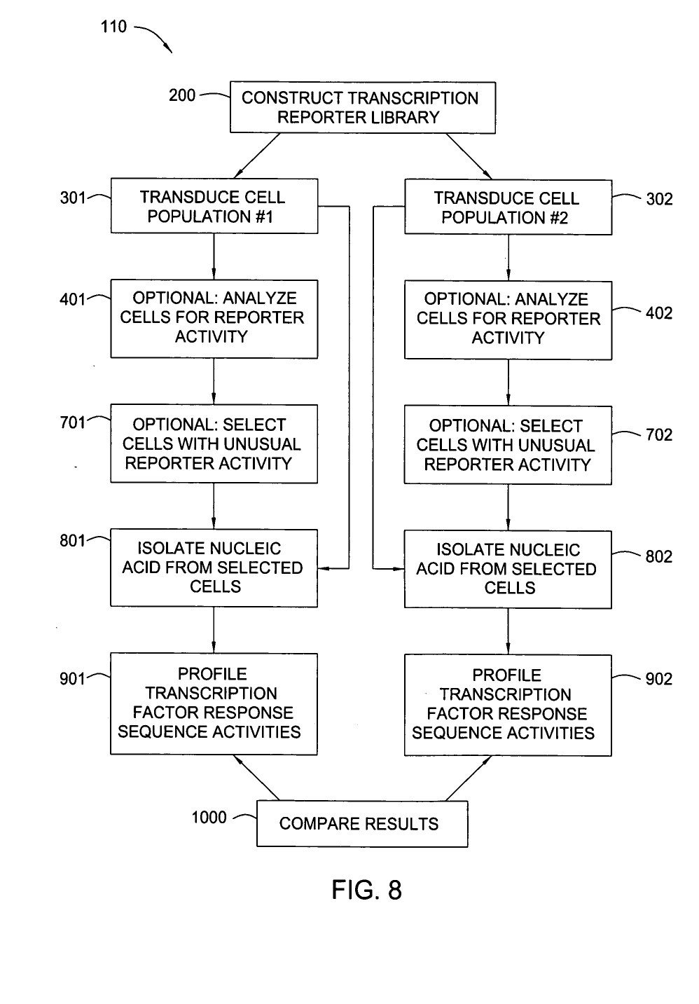

FIG. 8 is a block diagram of a simplified method 110 according to another embodiment of the present invention. In a first step, a transcription factor response reporter library is constructed (step 200). Next, two populations of target cells of interest are transduced with the transcription factor response reporter library (steps 301 and 302). The cell populations can be any cell populations of interest; for example, diseased and non-diseased cells, cancerous and non-cancerous cells, cells at different stages of development, cells that have been treated with an effector vs. cells that have not, and the like. In a preferred but optional step, the transduced populations of cells are then analyzed to determine reporter activity at steps 401 and 402; and cells from each population that demonstrate unusual reporter activity are selected (steps 701 and 702). At steps 801 and 802, nucleic acid is isolated from the selected cells from each population, at steps 901 and 902, the transcription factor response sequence responsible for the unusual reporter activity is identified. At step 1000, the results for each cell population are compared to identify the transcription factor response sequences that are affected in each cell population.

Transduction of the target cells with the pre-packaged viral effector library may be accomplished by methods known by those skilled in the art and depends generally on the target cell type and the viral vectors employed (step 300 of FIGS. 6 and 7 and steps 301 and 302 of FIG. 8). The target cells can be a pure, homogeneous population of the same or similar cells or the target cells can be a heterogeneous population of different cell types. The target cells may be cultured cells, or may be tissues, organs, biological fluids or whole organisms. Preferably, the organism is a human, mouse or rat.

The transduced target cells optionally are then analyzed to determine a baseline reporter activity (step 400 of FIGS. 6 and 7). One of the most flexible methods of analysis, particularly suitable to the methods of the present invention and useful with fluorescent reporters, is fluorescence-activated cell sorting (FACS). In preferred embodiments, the transcription factor response cassette drives a first reporter gene, such as the gene for a green fluorescent protein, and a second fluorescent reporter gene, such as a gene for a red fluorescent protein, under the control of a second promoter is used for a normalization control. FACS is employed to calculate the baseline ratio of, e.g., GFP/RFP (step 400 of FIG. 6). In the method shown in FIG. 8, FACS is employed to detect the average ratio of GFP/RFP fluorescence of each of the cell populations, as well as detect altered ratios in individual cells.

Once the baseline reporter activity is determined, the target cells may be treated with an effector (step 500 of FIGS. 6 and 7). The effector can be virtually anything that impact a cell, organ or organism, including but not limited to, environmental conditions such as temperature, growth media (salts, nutrients, etc.), aging, amount or wavelength of light, atmospheric conditions and the like; chemical compounds, such as vitamins, salts, carcinogens, toxic agents, drugs, polymers, proteins, peptides, enzymes, antibodies and the like; genetic agents, such as constructs expressing proteins, peptides, siRNAs, aptamers, ribozymes, sense and antisense RNAs delivered directly to the cell, organ or organism; and any other chemical genetic, or physical condition.

The treated cells are then analyzed again, preferably again by FACS, to both detect changes in the ratio of GFP/RFP fluorescence and select cells with an altered ratio (indicating a change in reporter activity) (steps 600 and 700 of FIGS. 6 and 7).

In the next step, 800 of FIGS. 6 and 7 (and steps 801 and 802 of FIG. 8), nucleic acids are isolated from the selected cells. Nucleic acid molecules may be prepared for analysis using any technique known to those skilled in the art. Preferably such techniques result in the production of a nucleic acid molecule sufficiently pure to identify the ID sequence in the selected cells. Such techniques may be found, for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell as genomic DNA, as it would be if the transcription factor response cassette and first reporter gene are configured as shown in FIGS. 2B or FIG. 2C and the vector is a lentiviral vector with a disabled 3/LTR, it may be necessary to first prepare an extract of the cell and then perform further steps—i.e., differential precipitation, column chromatography, matrix binding, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or chloroform to denature any contaminating and potentially interfering proteins. When chaotropic salts are used, it may be desirable to remove the salts from the nucleic acid-containing sample. This can be accomplished using standard techniques in the art such as precipitation, filtration, size exclusion chromatography, binding to a matrix such as glass particles or filters, and the like. Once isolated, the DNA is then amplified.

Alternatively, if the transcription factor response cassette and first reporter gene are configured as shown in FIG. 2A, it is desirable to extract and separate the messenger RNA from the separated target cells. Techniques and material for this purpose are known to those skilled in the art and may involve the use of oligo dT attached to a solid support such as a bead or plastic surface. It may be desirable to reverse transcribe the mRNA into cDNA using, for example, a reverse transcriptase enzyme. Suitable enzymes are commercially available from, for example, Invitrogen, Carlsbad, Calif. Optionally, the cDNA prepared from the mRNA is then amplified by protocols well-known in the art, such as PCR, T7 RNA polymerase linear amplification, strand displacement amplification, etc.

In preferred embodiments, the ID sequence (and, in some embodiments, the transcription factor response sequence) is amplified from the isolated DNA or cDNA before identification by sequencing or hybridization to a microarray. Nucleic acid amplification increases the number of copies of the nucleic acid sequence of interest. Any amplification technique known to those of skill in the art may be used in conjunction with the present invention including, but not limited to, polymerase chain reaction (PCR) techniques. PCR may be carried out using materials and methods known to those of skill in the art.

PCR amplification according to the present invention may involve the use of the isolated, single-stranded or double-stranded cDNA as a template. As such, the cDNA may be hybridized to a primer having a sequence complementary to a portion of the template sequence (such as one of the common sequences shown in FIG. 2D) and combined with a suitable reaction mixture including dNTPs and a polymerase enzyme. The primer is then elongated by the polymerase enzyme producing a nucleic acid complementary to the original template. For the amplification of both strands of a double-stranded nucleic acid molecule, two primers are used, each of which has a sequence complementary to a portion of one of the nucleic acid strands (such as both of the universal sequences shown in FIG. 2D). Elongation of the primers with a polymerase enzyme results in the production of double-stranded nucleic acid molecules each of which contains a template strand and a newly-synthesized complementary strand. The strands of the nucleic acid molecules are denatured—for example, by heating—and the process is repeated, this time with the newly-synthesized strands of the preceding step serving as templates in the subsequent steps. A PCR amplification protocol may involve a few to many cycles of denaturation, hybridization and elongation reactions to produce sufficient amounts of the desired nucleic acid.

Although PCR methods typically employ heat to achieve strand denaturation and allow subsequent hybridization of the primers, any other means that results in making the nucleic acids available for hybridization to the primers may be used. Such techniques include, but are not limited to, physical, chemical, or enzymatic means, for example, by inclusion of a helicase, (see Radding, Ann. Rev. Genetics 16: 405-436 (1982)) or by electrochemical means (see PCT Application Nos. WO 92/04470 and WO 95/25177). Template-dependent extension of primers in PCR is catalyzed by a polymerase enzyme in the presence of at least 4 deoxyribonucleotide triphosphates (typically selected from dATP, dGTP, dCTP, dUTP and dTTP) in a reaction medium which comprises the appropriate salts, metal cations, and pH buffering system. Suitable polymerase enzymes are known to those of skill in the art and may be cloned or isolated from natural sources and may be native or mutated forms of the enzymes. So long as the enzymes retain the ability to extend the primers, they may be used in the amplification reactions of the present invention.

The nucleic acids used in the methods of the invention may be labeled to facilitate detection in subsequent steps. Labeling may be carried out during an amplification reaction by incorporating one or more labeled nucleotide triphosphates and/or one or more labeled primers into the amplified sequence. The nucleic acids may be labeled following amplification, for example, by covalent attachment of one or more detectable groups. Any detectable group known to those skilled in the art may be used, for example, fluorescent groups, chemiluminescent groups, light scattering, electrochemical ligands and/or radioactive groups. In a preferred embodiment, biotin-labeled primers complimentary to regions flanking the ID sequence are used to amplify ID sequences from genomic DNA or cDNA by PCR. Then the amplified biotin-labeled probes are hybridized with a microarray and the bound fraction is detected on the array with phycoerythrin-labeled streptavidin or Cy3-labeled antibodies against biotin.

The preferred method of transcription factor response identification (step 900 of FIGS. 6 and 7 and steps 901 and 902 of FIG. 8) according to the present invention is by hybridizing the amplified ID sequences (or amplified ID/transcription factor response sequences) to a microarray. The transcription factor response sequences included in the viral reporter library are linked to the ID sequences that are included on the microarray or microarrays used for analysis. Briefly, a microarray is an ordered arrangement of probes, typically nucleic acids, on a solid or semi-solid support, where the probes have a defined location. Microarrays may have thousands or tens of thousands or more different probes. There are many microarrays and microarray vendors known in the art. For example, Affymetrix, Inc. (Santa Clara, Calif.), manufactures the GeneChip® by in situ photolithographic synthesis of approximately 10,000 to 1,200,000 25-mer oligonucleotides onto silicon wafers, which are then diced into chips. The readout or analysis is accomplished by fluorescence. Agilent, BDB Clontech, Amersham, and ABI provide about 10,000 500-5000-mer cDNAs or 4,000-44,000 45-80-mer oligonucleotides synthesized on the chip or printed by ink-jet printer or by pins onto glass slides, which are also analyzed by fluorescence, radioactivity or chemiluminescense. Essentially, microarrays can be made by photolithography, spotting oligonucleotides synthesized by standard phosphoramidite chemistry, photochemistry, electrochemistry, or the like. Analysis techniques include fluorescence, mass spectrometry, chemiluminescense or radioisotopic methods.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

All references cited herein are to aid in the understanding of the invention, and are incorporated in their entireties for all purposes.

1. A transcription reporter library comprising at least ten different transcriptional reporter vector constructs, wherein each transcriptional reporter vector construct comprises an identification sequence coupled to at least one transcription factor response element sequence operably coupled to a first promoter to produce transcription factor response cassettes, and wherein each transcription factor response cassette is different from one another and is operably coupled to first reporter sequence.

2. The transcription reporter library of claim 1, wherein the at least 10 different transcription reporter vector constructs further comprise a second reporter gene operably coupled to a second promoter.

3. The transcription reporter library of claim 2, wherein the second reporter sequence codes for a bioluminescent, chemiluminescent, or fluorescent protein.

4. The transcription reporter library of claim 2, wherein the second promoter is a constitutive promoter.

5. The transcription reporter library of claim 1, wherein the identification sequences are different from one another.

6. The transcription reporter library of claim 1, wherein the reporter vector is a viral vector.

7. The transcription reporter library of claim 6, wherein the viral vector is a retroviral vector.

8. The transcription reporter library of claim 6, wherein the viral vector is a lentiviral vector.

9. The transcription reporter library of claim 8, wherein the lentiviral vector is an FIV vector.

10. The transcription reporter library of claim 1, wherein the first reporter sequence codes for a bioluminescent, chemiluminescent, or fluorescent protein.

11. The transcription reporter library of claim 1, wherein the transcription factor response sequences comprise one to ten transcription response element sequences, attenuator sequences, enhancer sequences, silencer sequences or other sequences that regulate transcription.

12. The transcription factor response reporter library of claim 11, wherein the transcription factor response sequences comprise two or more copies of the same transcription response element sequence, attenuator sequence, enhancer sequence, silencer sequence or other sequence that regulates transcription, or a combination of one or more of any of transcription response element sequences, attenuator sequences, enhancer sequences, silencer sequences or other sequences that regulate transcription.

13. The transcription reporter library of claim 1, wherein the first promoter is regulated by transcription factors interacting with the transcriptional factor response sequence.

14. The transcription reporter library of claim 1, wherein the first promoter is a minimal promoter.

15. The transcription reporter library of claim 1, wherein the identification sequence is downstream of the first promoter.

16. The transcription reporter library of claim 6, wherein the transcription reporter library is packaged in viral particles.

17. A method for identification of sequences that affect transcription of a reporter gene in response to an effector comprising:
transducing target cells with the viral particles of claim 16;
treating the target cells with an effector;
analyzing the target cells for altered reporter activity;
selecting one or more target cells with the altered reporter activity; and
identifying the transcription factor response sequence present in the target cells, where the transcription factor response sequence controls the transcription of the reporter gene and is responsive to a transcription factor, which is in turn responsive to the effector.

18. A method for constructing a reporter cell line responsive to the effector used to treat the cells of claim 17, comprising transforming cells with a nucleic acid comprising the transcription factor response sequence identified in claim 17 operably coupled to a reporter sequence.

19. A method for analyzing differences in phenotype in at least two different cell populations comprising:
transducing the at least two different cell populations with the viral particles of claim 16;
analyzing the at least two cell populations for reporter activity;
selecting cell fractions with altered levels of reporter activity within each population;
identifying the transcription factor response sequence present in the selected cells from each cell population, where the transcription factor response sequence controls the transcription of the reporter gene and is responsive a transcription factor, which is in turn responsive to the phenotype; and
comparing the identified transcription factor response sequences affected in each cell population.

20. A method for constructing a reporter cell line with the phenotype of a population of the cells of claim 19, comprising transforming cells with a nucleic acid comprising the transcription factor response sequence identified in the population of cells in claim 19 operably coupled to a reporter sequence.

21. A transcription reporter library comprising at least ten different transcriptional reporter vector constructs, where each transcriptional reporter vector construct comprises at least one transcription factor response sequence operably coupled to a first promoter to produce transcription factor response cassettes, wherein each transcription factor response cassette is different from one another and is operably coupled to an identification sequence and a first reporter sequence.

22. The transcription reporter library of claim 21, wherein the at least 10 different transcription reporter vector constructs further comprise a second reporter gene operably coupled to a second promoter.

23. The transcription reporter library of claim 22, wherein the second reporter sequence codes for a bioluminescent, chemiluminescent, or fluorescent protein.

24. The transcription reporter library of claim 22, wherein the second promoter is a constitutive promoter.

25. The transcription reporter library of claim 21, wherein the identification sequences are different from one another.

26. The transcription reporter library of claim 21, wherein the reporter vector is a viral vector.

27. The transcription reporter library of claim 26, wherein the viral vector is a retroviral vector.

28. The transcription reporter library of claim 27, wherein the viral vector is a lentiviral vector.

29. The transcription reporter library of claim 28, wherein the lentiviral vector is an FIV vector.

30. The transcription reporter library of claim 21, wherein the first reporter sequence codes for a bioluminescent, chemiluminescent, or fluorescent protein.

31. The transcription reporter library of claim 21, wherein the transcription factor response sequences comprise one to ten transcription response element sequences, attenuator sequences, enhancer sequences, silencer sequences or other sequences that regulate transcription.

32. The transcription factor response reporter library of claim 31, wherein the transcription factor response sequences comprise two or more copies of the same transcription response element sequence, attenuator sequence, enhancer sequence, silencer sequence or other sequence that regulates transcription., or a combination of one or more of any of transcription response element sequences, attenuator sequences, enhancer sequences, silencer sequence or other sequences that regulate transcription.

33. The transcription reporter library of claim 21, wherein the first promoter is regulated by transcription factors interacting with the transcriptional factor response sequence.

34. The transcription reporter library of claim 21, wherein the first promoter is a minimal promoter.

35. The transcription reporter library of claim 26, wherein the transcription reporter library is packaged in viral particles.

36. A method for identification of sequences that affect transcription of a reporter gene in response to an effector comprising:
transducing target cells with the viral particles of claim 35;
treating the target cells with an effector;
analyzing the target cells for altered reporter activity;
selecting one or more target cells with the altered reporter activity; and
identifying the transcription factor response sequence present in the target cells, where the transcription factor response sequence controls the transcription of the reporter gene and is responsive to a transcription factor, which is in turn responsive to the effector.

37. A method for constructing a reporter cell line responsive to the effector used to treat the cells of claim 36, comprising transforming cells with a nucleic acid comprising the transcription factor response sequence identified in claim 36 operably coupled to a reporter sequence.

38. A method for analyzing differences in phenotype in at least two different cell populations comprising:
transducing at least two different cell populations with the viral particles of claim 35;
analyzing the at least two cell populations for reporter activity;
selecting cell fractions with altered levels of reporter activity within each population;
identifying the transcription factor response sequence present in the selected cells from each cell population, where the transcription factor response sequence controls the transcription of the reporter gene and is responsive a transcription factor, which is in turn responsive to the phenotype; and
comparing the identified transcription factor response sequences affected in each cell population.

39. A method for constructing a reporter cell line with the phenotype of a population of the cells of claim 38, comprising transforming cells with a nucleic acid comprising the transcription factor response sequence identified in the population of cells in claim 38 operably coupled to a reporter sequence.

40. A method for simultaneously assaying in the same reaction at least five transcription factors that affect transcription of a reporter:
obtaining at least five identification sequences;
coupling each of the at least five identification sequences to at least five different transcription factor response sequences to produce a set of transcription factor response cassettes;
ligating the set of transcription factor response cassettes into a reporter vector to produce transcription reporter constructs;
packaging the transcription reporter constructs into viral particles to produce a viral reporter library;
transducing target cells with the viral reporter library;
treating the target cells with an effector;
analyzing the target cells for altered reporter activity where altered reporter activity indicates a change in transcription;
selecting one or more target cells with the altered reporter activity;
isolating the identification sequence nucleic acid from the selected target cells; and
identifying the transcription factor response sequence present in the target cell, where the transcription factor response sequence controls the transcription of the reporter gene and is responsive to a transcription factor, which is in turn responsive to the effector.

41. The method of claim 40, further comprising a first analyzing step performed between the transducing step and the treating step.

42. The method of claim 40, further comprising an amplification step between the isolating step and the identifying step.

43. The method claim 40, wherein the identifying step is performed by hybridization of the isolated transcription factor response reporter construct to a microarray.

44. A method of constructing a packaged viral transcription reporter library comprising:
synthesizing a set of at least 100 different identification sequences on a glass substrate;
decoupling the at least 100 different identification sequences from the glass substrate;
synthesizing transcription factor response sequences on a glass substrate;
decoupling the transcription factor response sequences from the glass substrate;
coupling each identification sequence to at least one transcription factor response sequence to produce at least 100 different transcription factor response cassettes;
obtaining a reporter vector, wherein the reporter vector comprises viral sequences and at least one reporter sequence;
ligating the transcription factor response cassettes to the reporter vector to produce viral response constructs, such that the reporter vector's first reporter sequence is operably coupled to a first promoter and the transcription factor response cassette; and
packaging the viral transcription reporter construct in viral particles; thereby producing a packaged viral transcription reporter library.

45. A method for screening for transcription factors capable of altering the transcription level of a reporter gene comprising introducing into cells a transcription reporter library comprising at least ten different identification sequences each of which is coupled to at least one transcription factor response sequence operably coupled to a promoter and a reporter sequence; screening the cells for a cell or subset of cells exhibiting an altered level of reporter gene transcription, wherein the altered level of reporter gene transcription is due to the action of a transcription factor on the transcription factor response sequence.


SEQUENCE LISTING (1 sequence)

SEQ ID NO: 1 - DNA, 74 bp, Artificial Sequence
  Transcription factor response cassette
cctatcagtgattgcgtctacgttggaacgcgcctccgcagtggtaagaacaagagaacactgcccctacatct